(19) United States
(12) Patent Application Publication
(10) Pub. No.: US 2019/0336000 A1
(43) Pub. Date: Nov. 7, 2019

(54) ACOUSTIC-ELECTROMAGNETIC TOMOGRAPHY

(71) Applicant: Elwha, LLC, Bellevue, WA (US)

(72) Inventor: Yaroslav A. Urzhumov, Bellevue, WA (US)

(21) Appl. No.: 15/969,627

(22) Filed: May 2, 2018

(51) Int. Cl. A61B 5/00; A61B 8/13

(52) U.S. Cl. CPC A61B 5/0073; A61B 2562/0238; A61B 8/13; A61B 5/0097

(57) ABSTRACT

In one embodiment, a superlens is used to sub-diffraction-limit focus a magnetic field within a volume. A local magnetic field intensity maximum, or “hotspot,” is thereby created that is focused in two spatial directions substantially parallel to the superlens. The hotspot extends from the superlens through one or more coplanar layers of the volume. An electric field is superimposed over the magnetic field within the volume to be imaged. The superposition of electric and magnetic fields induces localized Lorentz forces. The modulation of the magnetic and/or electric field causes the portion of the volume in the hotspot to vibrate and emit acoustic signals at a frequency suitable for acoustic imaging. An acoustic transducer receives the emitted acoustic signals. The location from which the acoustic signals are emitted is constrained in two dimensions by the superlens. Time-gating the acoustic signals received from the hotspot is used to localize the received acoustic signals in the third dimension.

If an Application Data Sheet (ADS) has been filed on the filing date of this application, it is incorporated by reference herein. Any applications claimed on the ADS for priority under 35 U.S.C. §§ 119, 120, 121, or 365(c), and any and all parent, grandparent, great-grandparent, etc., applications of such applications are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 U.S.C. § 119(e) for provisional patent applications, for any and all parent, grandparent, great-grandparent, etc., applications of the Priority Application(s)). In addition, the present application is related to the “Related Applications,” if any, listed below.

### PRIORITY APPLICATIONS

NONE

### RELATED APPLICATIONS

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc., applications of the Priority Applications and the Related Applications, including any priority claims, is incorporated herein by reference to the extent such subject matter is not inconsistent herewith.

### TECHNICAL FIELD

This disclosure relates to acoustic-electromagnetic volumetric imaging. For example, magnetic fields and electric fields may induce localized Lorentz forces within a volume to cause the volume to produce detectable acoustic signals for imaging.

### BRIEF DESCRIPTION OF THE DRAWINGS

FIG. 1A illustrates a block diagram of an acoustic-electromagnetic volumetric imaging system, according to one embodiment.

FIG. 1B illustrates a magnetic field intensity maximum, or magnetic field hotspot, formed within a volume by the acoustic-electromagnetic volumetric imaging system of FIG. 1A.

FIG. 1C illustrates an electric field generator of the acoustic-electromagnetic volumetric imaging system of FIG. 1A permeating the volume with an electric field.

FIG. 1D illustrates the portion of the volume at the location of the magnetic field hotspot vibrating due to a time-dependent electromagnetic force induced by the electric and magnetic fields therein.

FIG. 1E illustrates an acoustic transducer of the acoustic-electromagnetic volumetric imaging system of FIG. 1A receiving an acoustic signal produced by the time-dependent electromagnetic force vibrating the portion of the volume at the location of the magnetic field hotspot.

FIG. 2 illustrates a block diagram of another embodiment of the acoustic-electromagnetic volumetric imaging system that utilizes a magnetic metamaterial superlens to produce a subwavelength-focused magnetic field within a volume.

FIG. 3A illustrates a magnetic coil producing a relatively unfocused magnetic field within a volume, according to one embodiment.

FIG. 3B illustrates a magnetic coil and a superlens operating in concert to produce a subwavelength-focused magnetic field to a greater depth, according to one embodiment.

FIG. 4A illustrates a three-dimensional representation of a subwavelength magnetic field hotspot, according to one embodiment.

FIG. 4B illustrates locations of two time-gated cross-section acoustic signal data captures from the magnetic field hotspot of FIG. 4A.

FIG. 4C illustrates cross-sectional views of the relative magnitude of the magnetic field at the first and second time-gated cross-sections of FIG. 4B.

FIG. 5 illustrates a method for acoustic-electromagnetic volumetric imaging, according to one embodiment.

FIG. 6 illustrates a functional block diagram of an acoustic-electromagnetic volumetric imaging control system, according to one embodiment.

### DETAILED DESCRIPTION

According to various embodiments, systems, apparatuses and methods are described herein that relate to acoustic-electromagnetic imaging. In one specific embodiment, a superlens is used to sub-diffraction-limit focus a magnetic field within a volume. A local field intensity maximum, or “hotspot,” is thereby created that is focused in two spatial directions substantially parallel to the superlens within the volume. The hotspot of intense magnetic field may extend through one or more coplanar layers of the volume relative to the superlens. The electric field permeating the volume is modulated at a modulating frequency.

The modulating electric field produces an electromagnetic force on material within the magnetic field hotspot. The electromagnetic force causes the material within the magnetic field hotspot to vibrate and emit an acoustic signal. The frequency of the acoustic signal may be selected based on the modulation frequency of the electric field and the frequency of the magnetic field (which may be quasistatic in some embodiments to avoid thermal heating of the volume). An acoustic transducer may receive the acoustic signals emitted from the vibrating portion of the volume within the magnetic field hotspot. The location from which the acoustic signals are emitted is constrained in two dimensions by the superlens and time-gating may be used to localize the received acoustic signals in the third dimension. Ultrasonic imaging techniques may be used to generate three-dimensional images of the portion of the volume (or two-dimensional images if desired).

The specific embodiment above represents just one possible combination of components. Numerous alternatives and variations are within the scope of this disclosure. More generally, any of a wide variety of adjustable magnetic field generators may be used to produce a magnetic field intensity with a maximum in at least one cross-section of a volume to be imaged (i.e., a magnetic field hotspot). The magnetic field hotspot may permeate multiple cross-sections of the volume to be imaged. The cross-sections may be defined as planes substantially orthogonal to the propagation direction of the magnetic field that forms the hotspot.

The location of the magnetic field intensity maximum for a given cross-section is defined relative to the field intensities of other locations of the same cross-section, not globally. That is, the field localization of a magnetic field intensity maximum of a cross-section is defined within the two-dimensional manifold of the cross-section itself, and not within the direction of magnetic field propagation. A magnetic field intensity maximum can be synonymously described as being at/in/on/within/of a cross-section of the volume, and each cross-section can have a unique magnetic field intensity maximum. The magnetic field intensity maximums of multiple cross-sections may be substantially concentric when the cross-sections are defined orthogonal to the propagation direction of the magnetic field intensity maximum.

An electric current source may superimpose an electric field on the magnetic fields within the volume. The combination of the electric and magnetic fields causes particles within the volume to vibrate and produce an acoustic signal. At least one of the electric and magnetic fields may be modulated or otherwise varied with time.

An acoustic transducer may receive the acoustic signal produced by the time-dependent electromagnetic force on the volume induced by the electric and magnetic fields. The magnetic field intensity from the adjustable magnetic field generator may permeate multiple cross-sections, all of which may vibrate due to the applied magnetic field and electric field. The cross-sections of the magnetic field hotspot may be defined perpendicular to the adjustable magnetic field generator. An electronic circuit may time-gate the output of the acoustic transducer in temporal correlation with time-dependent adjustments to the magnetic field intensity maximum produced by the adjustable magnetic field generator.

An electronic controller may control operation of the adjustable magnetic field generator, the electric current source, and the time-gating electronic circuit. For example, the electronic controller may synchronize operation of the various components to enable high-resolution acoustic data capture from acoustic signals generated from within the volume itself.

In various embodiments, the magnetic field may be subwavelength focused as a magnetostatic beam, or hotspot, within the volume. The electric field may be a relatively weak, unfocused electric field, modulated at a desired ultrasound frequency, that permeates the volume. Portions of the volume within the hotspot will vibrate with significant force due to the relatively high-magnitude magnetic field within the hotspot at a frequency corresponding to the modulation frequency of the relatively weak, unfocused electric field. Portions of the volume outside of the hotspot will vibrate with very little force due to the lack of strong magnetic or electric fields.

In various embodiments, the magnetic field intensity maximum produced by an adjustable magnetic field generator provides spatial bounds for imaging in two dimensions with a resolution corresponding to the diameter of the cross-section of the magnetic field intensity maximum. The time gating of the received acoustic signal by the electronic circuit provides a spatial bound for imaging in the third dimension. The resolution in the dimensions corresponding to the diameter of the cross section of the magnetic field intensity maximum (i.e., again, the magnetic field hotspot) may vary with respect to the dimension bounded by the time-gating. That is, the diameter of the magnetic field hotspot may vary based on the distance from the adjustable magnetic field generator. For example, in some embodiments the magnetic field intensity maximum may be a hotspot that decays exponentially (or as a power law) with respect to distance from the adjustable magnetic field generator. The combination of the electric and magnetic fields induces a localized Lorentz force on the cross sections of the magnetic field hotspot to cause them to vibrate.

The volume may comprise a medium that is electromagnetically opaque at microwave frequencies, making it difficult or impossible to image with conventional microwave imaging techniques. For instance, volumes that include or are enclosed (or even partially enclosed) by media that is opaque at microwave frequencies may not be readily imaged using traditional microwave imaging (e.g., mapping the steady-state permittivity and/or conductivity distribution of the volume). Similar problems exist for millimeter-wave, terahertz and optical (including infrared) imaging techniques when the volume comprises a medium that is opaque (strongly scattering, absorptive, or reflective) at the imaging frequencies. For ultrasonic imaging, volumes may not be readily imaged if they include or are encased by a medium that is acoustically opaque, strongly scattering, absorptive, resonant, or highly reflective.

Examples of such volumes are not uncommon. For instance, the volume may include or be partially encased by an electrically conductive medium and/or include one or more metal-rich boundaries. The volume may include water with dissolved ionic salts that are not easily imaged with traditional imaging techniques. In some instances, the volume may be biological in nature, comprising, for example, soft tissues and/or bone material. Traditional ultrasonic imaging approaches cannot adequately image soft tissue that is obscured by, encased, or partially encased by bone material. For example, traditional ultrasonic imaging approaches cannot adequately image lung tissue within the rib cage or brain tissue within the skull. As another example, traditional ultrasound imaging techniques based on focused ultrasound beams cannot adequately image lungs, due to the high scattering and reflectivity of the numerous tissue/air interfaces present there. In contrast, the presently described systems and methods allow for the effective “insertion” of an ultrasonic probe through the bone material into the soft tissue, and operating it at sufficiently low acoustic frequencies where acoustic attenuation is substantially lower than at the acoustic frequencies used in traditional techniques. An ultrasound transducer receives time-gated ultrasound from the soft tissue as if it were being emitted from a point source within the soft tissue.

Generally, conventional imaging systems utilize high frequency RF and/or high frequency ultrasound because the image resolution is highly dependent on wavelength. Thus, it is generally difficult or impossible to image volumes that are opaque at the relevant frequencies for a given imaging system. The use of lower frequencies in traditional systems is not possible because the image resolution would be too low.

As noted above, the image resolution of the presently described systems and methods is not dependent on the wavelength of the electric or magnetic fields. Rather, the resolution in the plane orthogonal to the direction of the magnetic field propagation is based on the time-gating intervals, and the resolution in the planar cross sections is based on the diameter of the magnetic field hotspot. For example, in a given time-gated cross-section the magnetic field hotspot may be centered at the local maximum of magnetic field within the plane and the diameter of the magnetic field hotspot may correspond to a defined falloff point of the magnetic field intensity within the plane (e.g., the 3 dB, 6 dB, or 10 dB falloff points). Because the resolution of the systems and methods described herein is not dependent on the wavelength of the electric and magnetic fields, high-resolution imaging is possible using relatively low frequencies.

The acoustic transducer may comprise one or more ultrasound microphones and/or ultrasound receivers. The adjustable magnetic field generator may produce a quasistatic magnetic field and the electric current source may comprise an alternating current source modulated at ultrasonic frequencies (e.g., frequencies greater than 20 kilohertz). For example, the alternating electric current source may be modulated at frequencies between 750 kilohertz and 2 megahertz. The modulation frequency may be selected to correspond to a target acoustic frequency to be received by the acoustic transducer. For instance, the electric current source may be modulated at a frequency many times greater than the quasistatic magnetic field (e.g., tens, hundreds, or even thousands of times greater).

The adjustable magnetic field generator may comprise an array of adjustable magnetic field sources. Each of the magnetic field sources may comprise, for example, an inductor and/or an adjustable array of static magnetic field sources. In some embodiments, the adjustable magnetic field source may comprise an array of electromechanically-actuated static magnetic field sources that are, for example, physically displaced to achieve magnetic field amplitude or direction modulation. In some embodiments, electromechanically-actuated static magnetic field sources are physically rotated during electromechanical actuation to achieve magnetic field amplitude, direction, or polarity modulation.

In some embodiments, the adjustable array of static magnetic field sources may be adjusted to maximize magnetic field two-dimensional localization in one particular cross section of the magnetic field hotspot. Static magnetic field sources, such as those used in an electromechanically adjustable array, may include permanent magnets, electromagnets, and/or superconducting coils.

The adjustable magnetic field generator may produce an alternating magnetic field at a frequency greater than, for example, 20 kilohertz. For instance, the alternating magnetic field may operate at 1 megahertz or more. In such embodiments, the electric current source may be a DC current source to generate a DC electric field within the volume to be imaged. Alternatively, an alternating current source may be used to generate an AC electric field. The alternating current source may operate at a frequency greater than that of the alternating magnetic field.

The electric current source may generate an RF electric field within the volume that, in combination with the magnetic field, induces localized Lorentz forces on particles within the volume to be imaged. The RF electric field may operate at any of a wide variety of frequencies including, for example, various frequencies of frequency bands between 1 kilohertz and 20 megahertz.

As described herein, an adjustable magnetic field generator may comprise a combination of magnetic field sources positioned relative to a magnetic metamaterial superlens. For example, a cylindrical superlens may be used to provide sub-diffraction-limited focus of the magnetic field to the hotspot. The magnetic metamaterial superlens may include magnetic metamaterial with a negative magnetic permeability for one or more polarizations of magnetic field. The magnetic permeability may be isotropic and negative for one or more polarizations of magnetic field.

A magnetic field source positioned relative to the magnetic metamaterial superlens may be moved (e.g., translated, moved farther or closer to the superlens, and/or rotated) relative to the magnetic metamaterial superlens in one or more directions to move and/or change the shape of the hotspot (i.e., the magnetic field intensity maximum). In some embodiments, the magnetic metamaterial superlens may be adjustable or tunable via, for example, electric or electromechanical input controls.

As previously described, the electric and magnetic fields cause material within the hotspot to vibrate and emit acoustic signals. An ultrasonic imaging system may process time-gated acoustic signals to generate an image of the hotspot based on images of a plurality of cross-sections. Multiple images, or one larger image (e.g., a stitched image), can be obtained by moving the hotspot within the volume and capturing additional time-gated images of the volume at each sequential hotspot location.

Many possible combinations of the embodiments and elements described above may be used to generate an image of a portion of a volume. In one specific embodiment, a magnetic field source and a magnetic metamaterial superlens operate in conjunction to produce a magnetic field intensity as a “hotspot” within a volume. An electric current source generates an electric field within the volume. The electric field may be focused to the hotspot or unfocused; either way, within the hotspot, the magnetic field and electric field induce localized time-dependent electromagnetic forces on the volume (e.g., localized Lorentz forces) that causes particles within the hotspot to vibrate and emit an acoustic signal.

An acoustic transducer receives the acoustic signals. The electromagnetic force on the volume induced by the electric and magnetic fields is time-dependent. Accordingly, an electronic circuit may time-gate the output of the acoustic transducer in temporal correlation with adjustments to the magnetic field intensity maximum. An electronic controller can control the operation of the electrical current source, adjustability and/or tunability of the superlens, the time-gating electronic circuit, and/or the magnetic field source.

As previously described, the electric current source may be a DC current source to generate a DC electric field within the volume, or it may be an AC current source to generate an AC electric field within the volume. The electric and magnetic fields may operate at any of a wide range of frequencies that may be selected to target a specific frequency of acoustic signal. For example, a target acoustic ultrasonic signal between 1 and 3 megahertz may be obtained by selecting corresponding electric and magnetic fields. Various harmonics and/or beat frequencies may be utilized and/or targeted in some embodiments.

In some embodiments, the acoustic transducer may comprise a single microphone that is coaxially located relative to an axis of the superlens and the generated hotspot. In other embodiments, the acoustic transducer may comprise an array of ultrasound receivers and/or may be non-coaxially located relative to the superlens.

The specific components and arrangements of the systems described herein are merely examples of possible embodiments. Various alternative systems, devices, and components may be utilized to induce an electromagnetic force within a volume to cause it to vibrate. A wide variety of systems and methods may be utilized to capture and localize the acoustic signals generated by the vibrations. Ultrasonic imaging techniques may be used to generate an image of the portion of the volume that is vibrating.

It is appreciated that alternative combinations of systems and components may be used to generate a magnetic field intensity maximum and an electric field within a volume, and then receive acoustic signals produced by the resulting electromagnetic force at the location of the magnetic field intensity maximum. The received acoustic signals may be used to generate ultrasonic images using, for example, time-gating techniques while controlling the generation of the magnetic and/or electric fields.

Thus, the presently described systems and methods utilize electric and magnetic fields to induce portion of a volume to emit ultrasound that can be imaged. This allows for imaging of volumes that cannot be readily imaged by traditional RF-based and Ultrasound-based imaging systems. The presently described systems and methods provide for non-invasive imaging in a way that, in many ways, is the functional equivalent of inserting a high-resolution ultrasound emitting probe into a precise location within a volume. As previously noted, volumes containing imaging-opaque media at the frequencies used by traditional imaging system may not be readily imaged by such systems. In contrast, the presently described systems and methods operate to “insert” a high-resolution acoustic probe (e.g., ultrasound probe) into the volume in a non-invasive way. The systems and methods enable the effective “insertion” into volumes containing or encased by media that does not otherwise led itself to imaging by traditional means.

In one specific embodiment, the magnetic field intensity maximum is a quasistatic magnetic field and the electric field comprises an alternating current electric field operating at or modulated at frequencies greater than 20 kilohertz to induce ultrasonic (i.e., greater than 20 kilohertz) emissions from the volume. For example, the alternating electric current source may be modulated at a frequency greater than 1 megahertz. By reducing the frequency of the magnetic field (e.g., by utilizing a quasistatic magnetic field) thermal heating of the volume may be reduced while retaining the ability to induce an electromagnetic force within a target region of the volume. Thus, unlike many systems in which an electromagnetic field comprises electric field and magnetic field components at the same frequency, many of the presently described embodiments contemplate disassociated electric and magnetic fields in the near-field that can be independently controlled, modulated, focused, etc.

The generalized descriptions of the systems and methods herein may be utilized and/or adapted for utilization in a wide variety of industrial, commercial, and personal applications. For example, the systems and methods described herein may be utilized in any of a wide variety of medical, security, and other imaging environments.

Many existing computing devices and infrastructures may be used in combination with the presently described systems and methods. Some of the infrastructure that can be used with embodiments disclosed herein is already available, such as general-purpose computers, computer programming tools and techniques, digital storage media, and communication links.

Moreover, the systems and methods described herein may utilize various technologies and principles from other fields, including, for example, those relating to magnetic resonances, sub-diffraction-limit focusing, superlenses, near-field focusing, metamaterial superlenses, near-field magnetic coupling for wireless power transfer, and the like. Examples of related technologies are found in the following disclosures, which are hereby incorporated by reference in their entities: Dmitriy Korobkin, et al. Enhanced Near-Field Resolution in Midinfrared Using Metamaterials, Journal of Optical Society of America B, Vol. 23, No. 3, March 2006, 468-478; Guy Lipworth, et al. Magnetic Metamaterial Superlens for Increased Range Wireless Power Transfer, Scientific Reports, 4:3642, www.nature.com/scientificreports, 10 Jan. 2014, 1-6; Da Huang, et al. Magnetic Superlens-Enhanced Inductive Coupling for Wireless Power Transfer, Journal of Applied Physics 111, 20 Mar. 2012, 064902-1-8; Jian-Wen Dong, et al. Metamaterial Slab as a Lens, a Cloak, or an Intermediate, Physical Review B 83, 15 Mar. 2011, 115124, 1-7; Yaroslav Urzhumov, et al. Metamaterial-Enhanced Coupling Between Magnetic Dipoles for Efficient Wireless Power Transfer, Physical Review B 83, 18 May 2011, 205114, 1-10; Manuel J. Freire, et al. On the Applications of μᵣ=−1 Metamaterial Lenses for Magnetic Resonance Imaging, Journal of Magnetic Resonance, http://alojamientos.us.es/gmicronda/Miembros/Freire/freire.htm, 2009; Thomas Taubner, et al., Near-Field Microscopy Through a SiC Superlens, Science Magazine Vol. 313, 15 Sep. 2006, 1595.

Many of the embodiments described herein can be implemented with metamaterial devices. Metamaterial devices can be used to form lens, such as superlenses, that have negative permeability and/or negative permittivity properties for at least some wavelengths of electromagnetic radiation. Examples of negative-permeability/permittivity metamaterials and uses thereof are described in the following disclosures, each of which is hereby incorporated by reference in its entirety: U.S. patent application Ser. No. 11/355,493 titled “Variable Metamaterial Apparatus” filed on Feb. 16, 2006; U.S. patent application Ser. No. 10/525,191 titled “Indefinite Materials” filed on Aug. 29, 2003; U.S. patent application Ser. No. 11/658,358 titled “Metamaterials” filed on Jul. 22, 2005; and Victor Veselago, et al. Negative Refractive Index Materials, Journal of Computational and Theoretical Nanoscience, Vol. 3, 1-30, 2006.

A computing device or controller may include a processor, such as a microprocessor, a microcontroller, logic circuitry, or the like. A processor may include one ore more special-purpose processing devices, such as application-specific integrated circuits (ASICs), programmable array logic (PAL), programmable logic array (PLA), programmable logic device (PLD), field-programmable gate array (FPGA), or other customizable and/or programmable device. The computing device may also include a machine-readable storage device, such as non-volatile memory, static RAM, dynamic RAM, ROM, CD-ROM, disk, tape, magnetic, optical, flash memory, or another machine-readable storage medium. Various aspects of certain embodiments may be implemented using hardware, software, firmware, or a combination thereof.

The components of the disclosed embodiments, as generally described and illustrated in the figures herein, could be arranged and designed in a wide variety of different configurations. Furthermore, the features, structures, and operations associated with one embodiment may be applied to or combined with the features, structures, or operations described in conjunction with another embodiment. In many instances, well-known structures, materials, or operations are not shown or described in detail to avoid obscuring aspects of this disclosure.

The embodiments of the systems and methods provided within this disclosure are not intended to limit the scope of the disclosure but are merely representative of possible embodiments. In addition, the steps of a method do not necessarily need to be executed in any specific order, or even sequentially, nor do the steps need to be executed only once. Some aspects of the various embodiments are illustrated in the drawings and additional understanding can be gained therefrom.

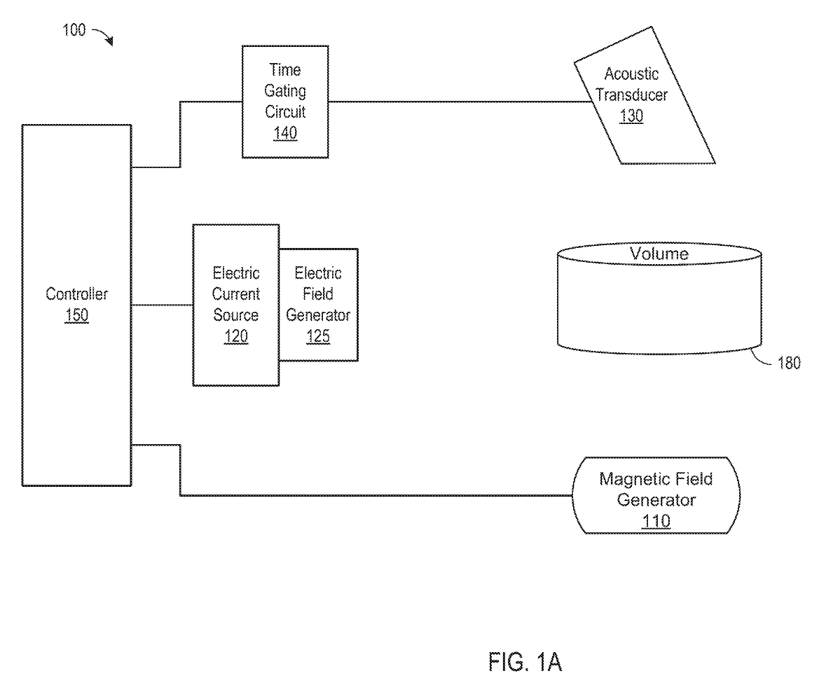

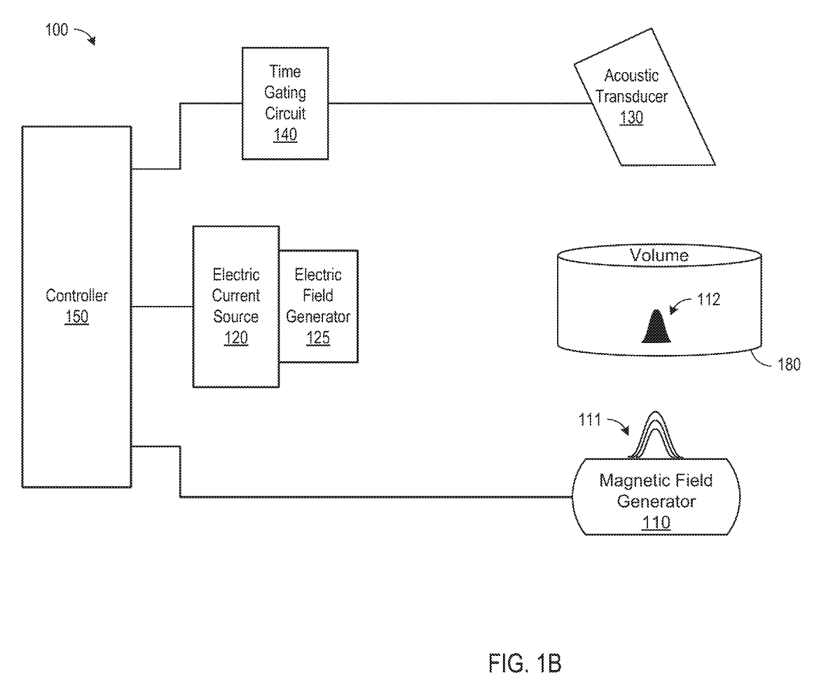

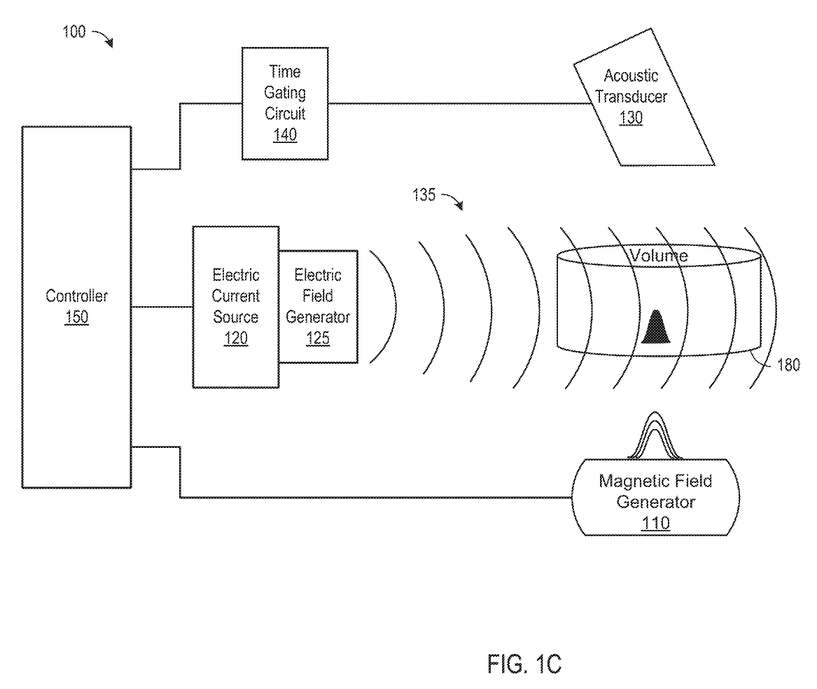

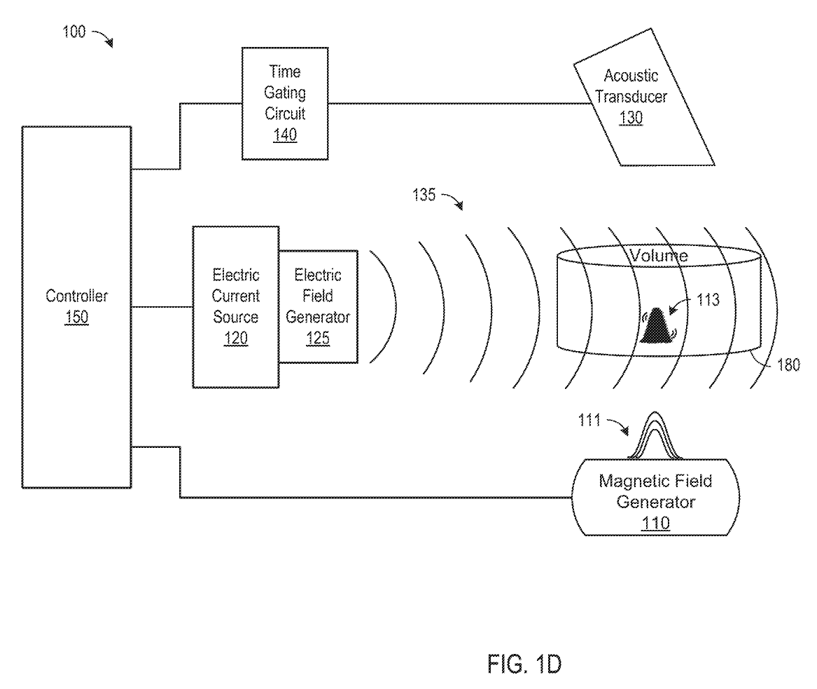

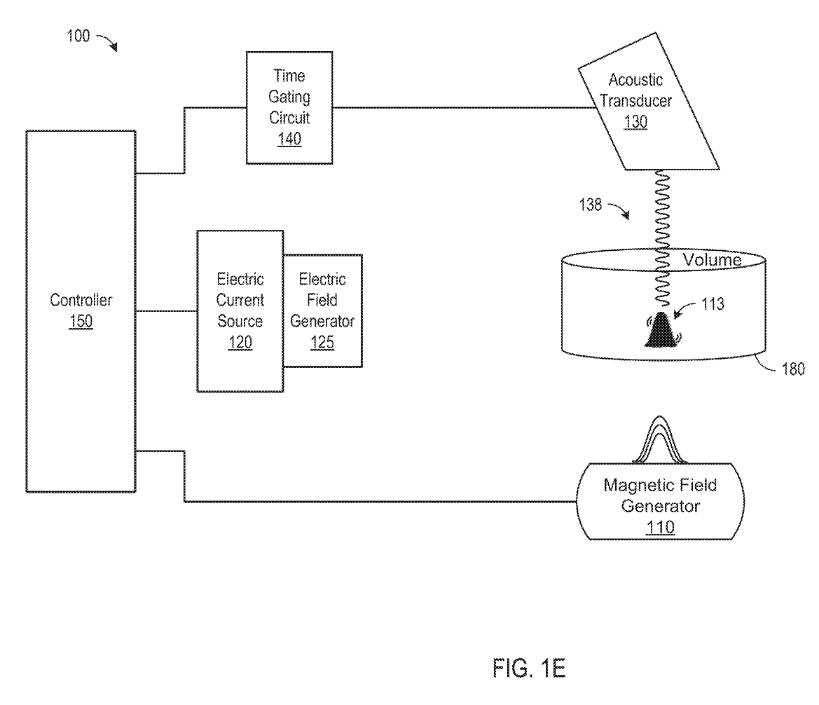

FIG. 1A illustrates a block diagram of an acoustic-electromagnetic volumetric imaging system 100, according to one embodiment. The acoustic-electromagnetic volumetric imaging system 100 may include a magnetic field generator 110 to produce a magnetic field intensity maximum in one or more cross-sections of the volume 180. As an example, the magnetic field intensity maximum may comprise a magnetic field hotspot formed within the volume 180.

The magnetic field generator 110 may, for example, comprise an array of adjustable or static magnetic field sources. In other embodiments, the magnetic field generator 110 may comprise a magnetic metamaterial superlens and at least one magnetic field source positioned relative to the magnetic metamaterial superlens. The magnetic field generator 110 may produce a low frequency or even a quasistatic magnetic field in various embodiments. in other embodiments, the magnetic field generator 110 may produce a relative high frequency or non-quasistatic magnetic field at a frequency greater than 20 kilohertz (e.g., 20 kilohertz, 1 megahertz, 3 megahertz, 10 megahertz, etc.).

An electric current source 120 and an associated electric field generator 125 may generate a focused or unfocused electromagnetic field (e.g., an RF field) that permeates the volume 180. In embodiments in which the magnetic field operates at frequencies greater than 20 kilohertz, the electric current source 120 and associated electric field generator 125 may produce a relatively low frequency or even a direct current (DC) electric field. In other embodiments, the magnetic field generator 110 may produce a low frequency or quasistatic magnetic field and the electric current source 120 and associated electric field generator 125 may produce a relatively high-frequency electric field (e.g., 100 kilohertz, 1 megahertz, between 5 and 10 megahertz, etc.).

In still other embodiments, both the electric and magnetic fields may operate at non-zero frequencies. In some such embodiments, the electric current source 120 may be modulated at a frequency of at least two times greater than the frequency of an alternating magnetic field generated by the magnetic field generator 110.

An acoustic transducer 130, or an array of acoustic transducers, may receive acoustic signals (e.g., ultrasonic acoustic signals) generated by vibrations of a portion of the volume 180 at a location of the magnetic field hotspot due to electromagnetic forces on particles within the magnetic field hotspot. The electromagnetic forces, such as Lorentz forces, are forces exerted on particles within the magnetic field hotspot that oscillate back and forth due to the mathematical cross of the electric and magnetic fields, at least one of which is an alternating field. The total force, F, on a particle is given by Equation 1 below.

F=qE+qv×B Equation 1

As shown above, the total applied electromagnetic force vector, F, is a vector sum of the electric force component, qE, also known as the Coulomb force, and the magnetic force component, qv×B, also known as the Lorentz force, where E is the electric field vector, q is the charge of the particle, v is the velocity vector of the particle, and B is the magnetic field vector. As shown above, the magnetic force component, qv, is the vector cross-product of the velocity vector, and wherein the force on a particle is based on the charge, q, of a particle, the magnitude, E, of the electric field, the vector cross-product of the velocity, v, and the magnetic field vector, B. In the continuum description of a conductive medium, Equation 1 is replaced with its continuum equivalent:

F=ρE+J×B=ρE+σE×B Equation 2

In Equation 2, F is the volumetric force density, ρ is the (electric) charge density, J is the (electric) current density, and σ is the electrical conductivity of the medium, assumed to be an isotropic scalar for example. The Lorentz force portion of this force is then proportional to the cross-product of electric and magnetic fields.

Thus, in some embodiments, the magnetic field generator 110 may focus a relatively high-amplitude magneto static beam, or hotspot, within the volume 180. The electric field generator 125 may generate a relatively weak, unfocused electric field that permeates the volume 180 modulated at a desired ultrasound frequency.

Portions of the volume 180 within the hotspot will vibrate with significant force due to the relatively high-magnitude magnetic field within the hotspot at a frequency corresponding to the modulation frequency of the relatively weak, unfocused electric field. Portions of the volume 180 outside of the hotspot will vibrate with very little force due to the lack of strong magnetic or electric fields.

Acoustic signals are emitted from the portions of the volume 180 within the hotspot due to the vibrations at the modulation frequency of the electric field. The acoustic transducer 130 receives the acoustic signals. The acoustic signals may be generated at a target ultrasonic frequency by selecting a corresponding electric field modulation frequency. A time-gating circuit 140 may time-gate the received acoustic signals to localize the received acoustic signals within the volume 180. Generally speaking, acoustic signals from portions of the hotspot closest to the acoustic transducer 130 will be received before acoustic signals from portions of the hotspot farther from the acoustic transducer 130. Thus, the subwavelength-focused magnetic field provides for localization of the received acoustic signals in two dimensions while time-gating the received acoustic signals provides for localization in the third dimension. A controller 150 may control, e.g., synchronize, the operation of the magnetic field generator 110, electric current source 120, electric field generator 125, acoustic transducer 130, and/or the time-gating circuit 140.

FIG. 1B illustrates the magnetic field generator 110 of the acoustic-electromagnetic volumetric imaging system 100 of FIG. 1A generating a subwavelength-focused magnetic field 111 to produce a magnetic field hotspot 112 within the volume 180. As described herein, the magnetic field hotspot 112 may comprise a region within the volume 180 with a relatively high-intensity static or quasistatic magnetic field. For instance, the magnetic field may operate at less than 20 kilohertz (e.g., 10 kilohertz). In such an embodiment, Joule heating of the volume 180 is reduced because the relatively low-frequency magnetic field generator 110 reduces induced secondary electric fields within the volume 180.

FIG. 1C illustrates the electric field generator 125 of the acoustic-electromagnetic volumetric imaging system 100 of FIG. 1A permeating the volume 180 with an electric field 135. As previously described, the electric field 135 may be relatively weak and unfocused. The electric field 135 may be modulated at a relatively high frequency corresponding to a target acoustic signal to be received by the acoustic transducer 130.

FIG. 1D illustrates the portion of the volume 180 at the location of the magnetic field hotspot 113 of the acoustic-electromagnetic volumetric imaging system 100 of FIG. 1A vibrating due to a time-dependent electromagnetic force (e.g., a Lorentz force) induced by the electric field 135 and magnetic field 111 therein. As previously described, portions of the volume 180 within the location of the magnetic field hotspot 113 are vibrated with significant force due to the high-intensity magnetic field within the hotspot 113, while portions of the volume 180 outside of the magnetic field hotspot 113 do not vibrate or vibrate with minimal force due to weak electric and magnetic fields in those regions.

FIG. 1E illustrates the acoustic transducer 130 of the acoustic-electromagnetic volumetric imaging system 100 of FIG. 1A receiving an acoustic signal 138 produced by the time-dependent electromagnetic force vibrating the portion of the volume 180 at the location of the magnetic field hotspot 113. The acoustic signals originating from within the magnetic field hotspot 113 may be localized in one dimension based on the different distances between portions of the magnetic field hotspot 113 and the acoustic transducer 130 by the time-gating circuit 140.

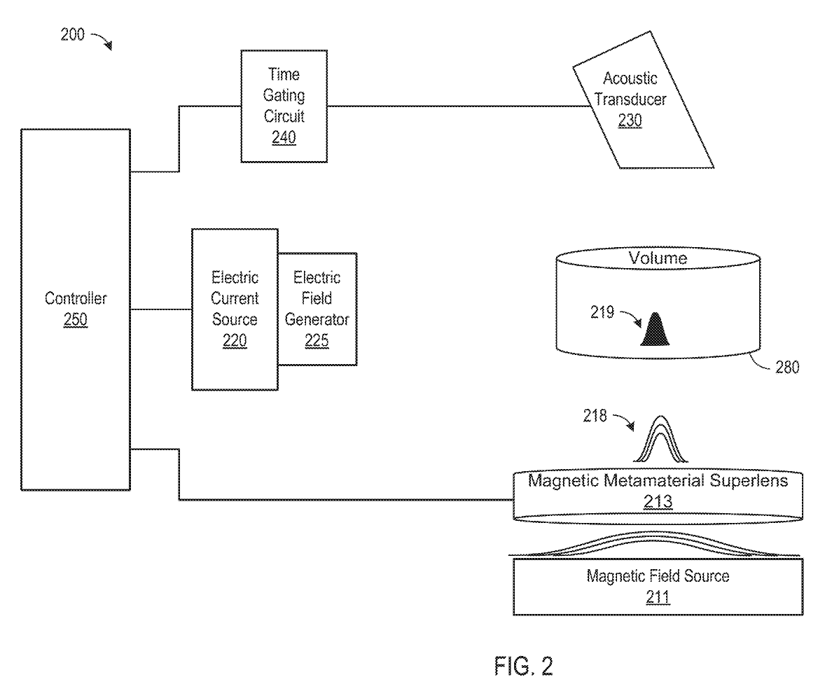

FIG. 2 illustrates a block diagram of another embodiment of the acoustic-electromagnetic volumetric imaging system 200 that utilizes a magnetic metamaterial superlens 213 to produce a subwavelength-focused magnetic field 218 to form a magnetic field hotspot 219 within a volume 280. The portion of the volume 280 within the magnetic field hotspot 219 may be vibrated to emit an acoustic signal corresponding to the vector sum of the frequencies of the magnetic field 218 and an electric field produced by the electric field generator 225.

In various embodiments, the magnetic field 218 may be selected to operate at a relatively low frequency (e.g., 10 kilohertz or less) to reduce Joule heating by induced secondary electric fields within the volume 280. The controller 250 may cause the electric current source 220 to drive the electric field generator 225 at a modulating frequency based on a target acoustic signal to be originated by the vibration of the portion of the volume 280 within the magnetic field hotspot 219.

The acoustic transducer 230 is configured to receive acoustic signals originated by the vibrating portion of the volume 280 within the magnetic field hotspot 219. The time-gating circuit 240 provides spatial localization of the received ultrasound in one dimension while the perimeter of each time-gated cross-section of the magnetic field hotspot 219 provides spatial localization in the other two dimensions. The controller 250 may synchronize the operation of the magnetic field source 211, the electric current source 220, the electric field generator 225, the acoustic transducer 230, and/or the time-gating circuit 240.

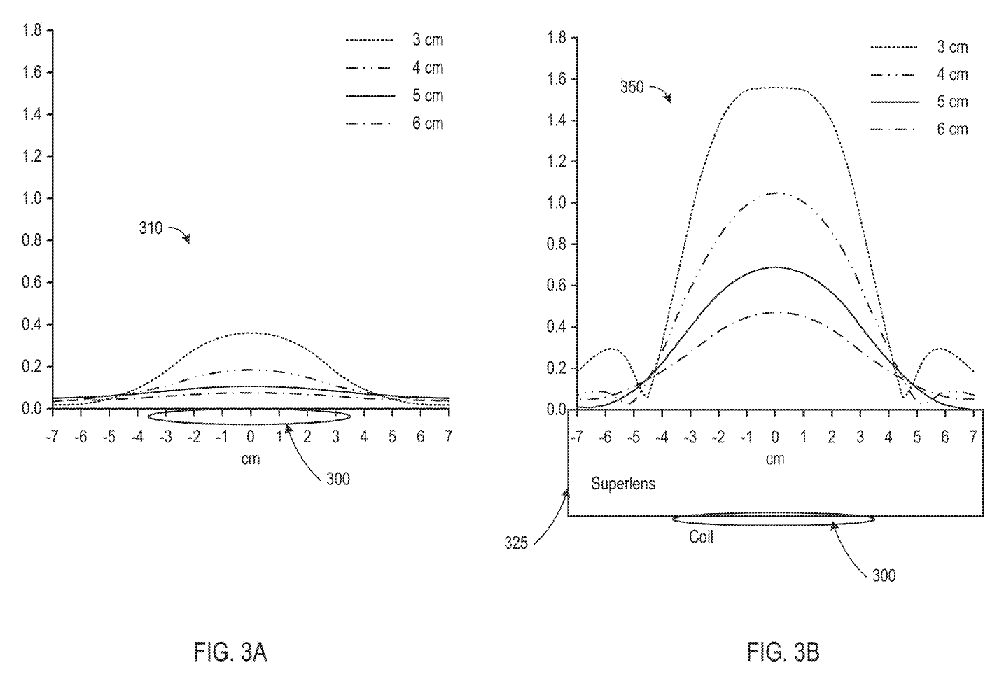

FIG. 3A illustrates a magnetic coil 300 producing a relatively unfocused magnetic field 310 within a volume, according to one embodiment. A relatively low-frequency magnetic field has a relatively large wavelength that cannot be subwavelength-focused using conventional beamforming. As shown, the relative magnitudes of the magnetic field at various depths is relatively low.

FIG. 3B illustrates the magnetic coil 300 and a superlens 325 operating in concert to produce a subwavelength-focused magnetic field 350 with relatively high magnitudes at greater depths into the volume, according to various embodiments. In various embodiments, the acoustic-electromagnetic volumetric imaging system leverages the subwavelength magnetic field abilities of the superlens to produce a magnetic field intensity maximum, or hotspot, within a volume. In some embodiments, a super lens may also be used to focus an electric field, while in other embodiments, the electric field may be unfocused.

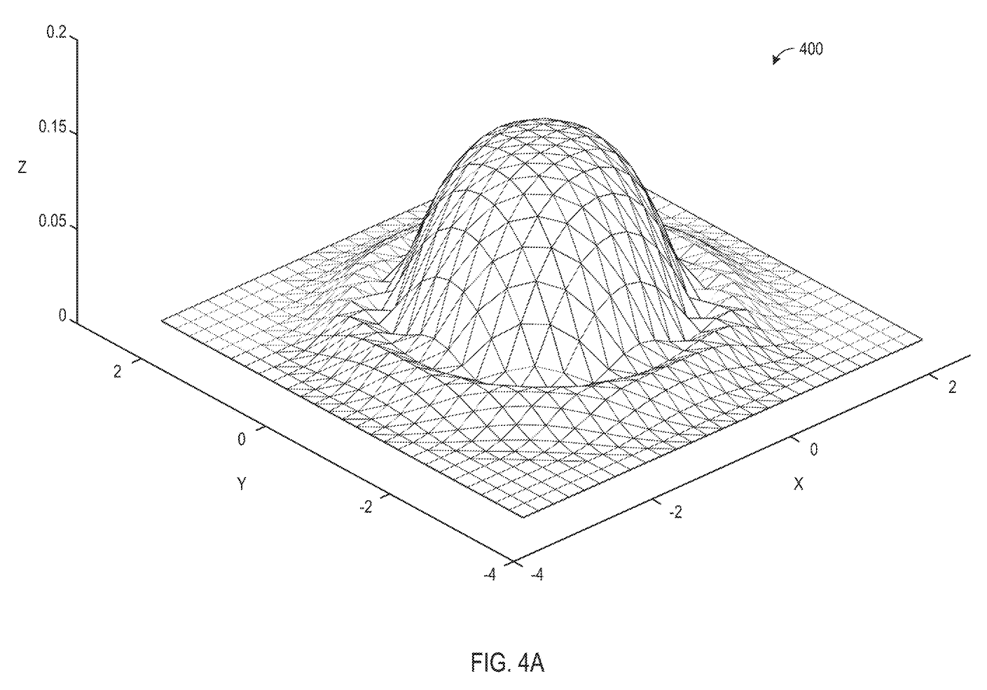

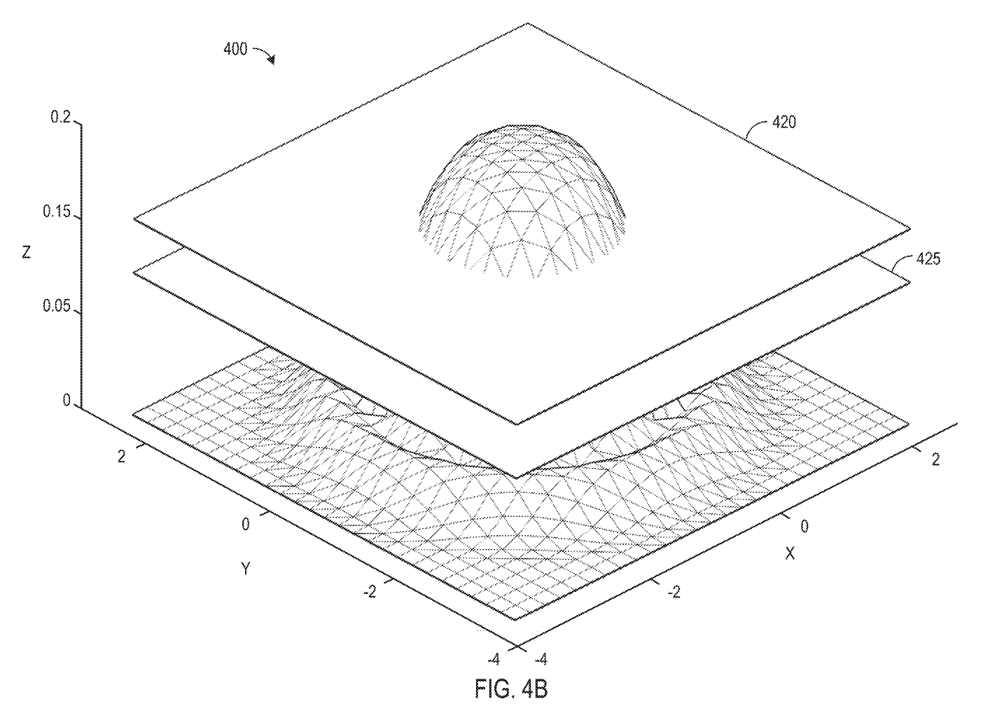

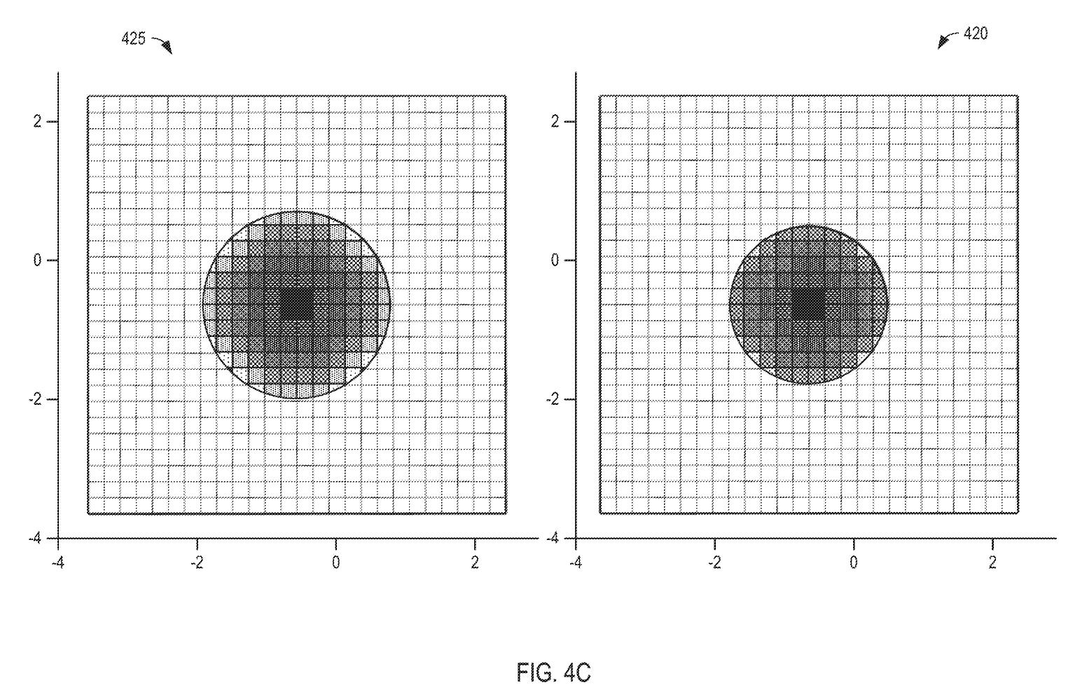

FIG. 4A illustrates a three-dimensional representation of a subwavelength magnetic field hotspot 400 within a volume, according to one embodiment.

FIG. 4B illustrates locations of two time-gated cross-sections 420 and 425 of acoustic signal data captured from the magnetic field hotspot of FIG. 4A. Spatial localization and corresponding resolution is based on the thickness of each of the time-gated cross-sections 420 and 425, which corresponds to the length of the time window during which acoustic signals are received for each of the time-gated cross-sections 420 and 425.

FIG. 4C illustrates time-gated cross-sections 425 and 420 from the corresponding time-gated cross-sections identified in FIG. 4B. As illustrated, the received acoustic signals are spatially localized in the other two dimensions based on the areas of the cross-sections 425 and 420 of the subwavelength magnetic field hotspot of FIG. 1A. As illustrated, the diameter of each of the time-gated cross-sections 425 and 420 may be different based on the relative depth into the volume from which they are captured.

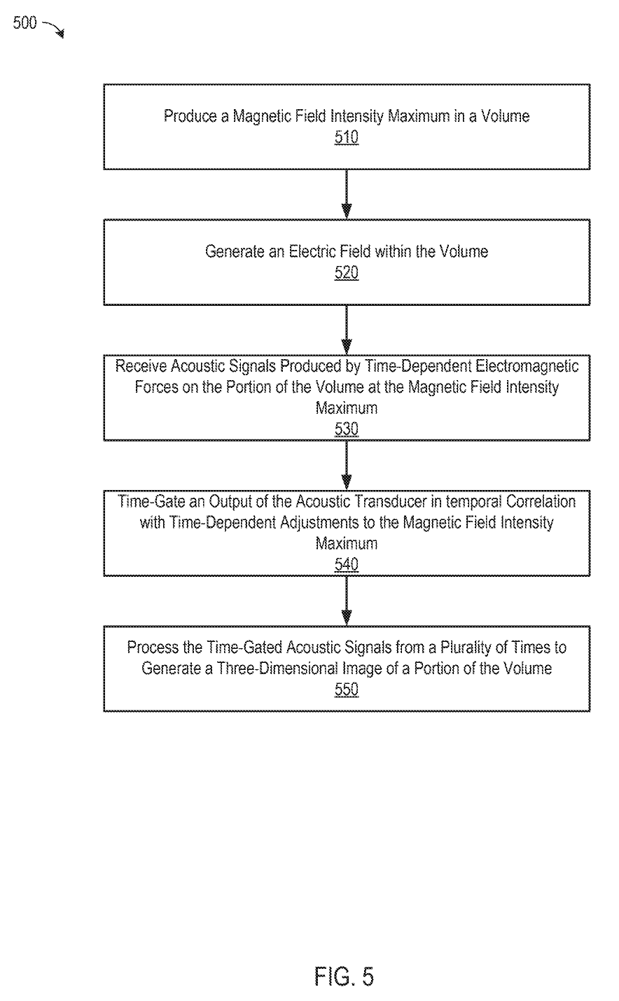

FIG. 5 illustrates a method 500 for acoustic-electromagnetic volumetric imaging, according to one embodiment. A magnetic field generator (e.g., a magnetic field source and superlens, or an array of magnetic field coils) may produce, at 510, a high-intensity magnetic field intensity maximum, or magnetic field hotspot, within a portion of a volume. An electric field generator may generate, at 520, an electric field within at least the portion of the volume at the location of the magnetic field hotspot. For example, the electric field may be weak and relatively unfocused. One or both of the magnetic field and the electric field may be modulated at frequencies corresponding to a target acoustic emission frequency.

The combination of the electric field and the magnetic field cause the portion of the volume at the location of the magnetic field hotspot to vibrate and emit acoustic signals. An acoustic transducer may receive, at 530, acoustic signals produced by the time-dependent electromagnetic forces on the portion of the volume at the magnetic field intensity maximum. A time-gating circuit may time-gate, at 540, an output of the acoustic transducer (or transducer array) in temporal correlation with the time-dependent adjustments to the magnetic field intensity maximum.

For example, the magnetic field intensity maximum may be positioned at a first location within the volume. Time-gated acoustic signals may be captured from the magnetic field intensity maximum positioned at the first location within the volume. The magnetic field intensity maximum may be moved to any number of additional locations within the volume by, for example: moving the volume relative to the acoustic-electromagnetic volumetric imaging system, moving or rotating the magnetic field generator, moving or rotating a superlens, and/or repositioning the acoustic transducer. Time-gated acoustic signals may be captured from each of the various locations of the magnetic field intensity maximums.

The plurality of captured time-gated acoustic signals from the various locations may be processed, at 550, to generate a three-dimensional image of various portions of the volume. For example, a subwavelength magnetic field hotspot may be raster-scanned within a volume permeated by a weak, unfocused electric field. Emitted acoustic signals from each location of the raster-scanned magnetic field hotspot may be captured and time-gated. The time-gated acoustic data from each location of the raster-scanned magnetic field hotspot may be processed to generate a three-dimensional image of the entire volume.

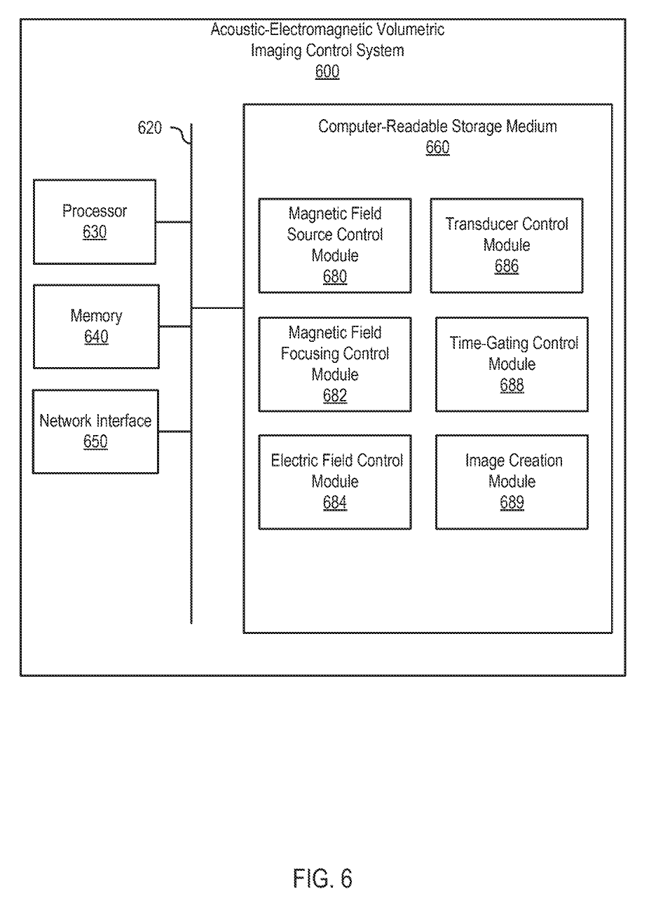

FIG. 6 illustrates a functional block diagram of an acoustic-electromagnetic volumetric imaging control system 600, according to one embodiment. The acoustic-electromagnetic volumetric imaging control system 600 may include a processor 630, memory 640, and, optionally, a network interface 650. A bus 620 may connect the memory 640 and processor 630 to a computer-readable storage medium 660, such as a non-transitory computer-readable storage medium. The computer-readable storage medium 660 may include a plurality of modules 680-689 executable by the processor 630 for implementing operations for acoustic-electromagnetic volumetric imaging.

A magnetic field source control module 680 may provide instructions for the processor 630 to control the generation of a magnetic field by a magnetic field source to produce a subwavelength magnetic field hotspot within a volume. A magnetic field focusing control module 682 may provide instructions for the processor 630 to control an adjustable array of magnetic field sources (e.g., an array of magnetic field coils) or an adjustable or tunable magnetic metamaterial superlens.

An electric field control module 684 provides instructions for the processor 630 to control the generation and modulation of an electric field by an electric field source. A transducer control module 686 provides instructions for the processor 630 to control the reception of acoustic signals by an acoustic transducer or array of acoustic transducers. A time-gating control module 688 provides instructions for the processor 630 to control time-gating of received acoustic signals. An image creation module 689 provides instructions for the processor 630 to generate a three-dimensional image based on time-gated acoustic signals from one or more magnetic field hotspots at one or more locations at one or more times within a volume.

In various embodiments, one or more of the modules 680-689 and/or the associated functionalities may be omitted from the acoustic-electromagnetic volumetric imaging control system 600. Moreover, one or more of the modules 680-689 may be separated into distinct sub-modules, combined into a single module, and/or implemented by a different processor or system.

This disclosure has been made with reference to various exemplary embodiments, including the best mode. Those skilled in the art will recognize that changes and modifications may be made to the exemplary embodiments without departing from the scope of the present disclosure. While the principles of this disclosure have been shown in various embodiments, many modifications of structure, arrangements, proportions, elements, materials, and components may be adapted for a specific environment and/or operating requirements without departing from the principles and scope of this disclosure. These and other changes or modifications are intended to be included within the scope of the present disclosure.

This disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, required, or essential feature or element. This disclosure should, therefore, be determined to encompass at least the following claims.

1. An acoustic-electromagnetic volumetric imaging system, comprising:
an adjustable magnetic field generator to produce a magnetic field intensity having a maximum in at least one cross-section of a volume to be imaged;
an electric current source to generate an electric field within the volume;
an acoustic transducer to receive acoustic signals produced within the volume by a time-dependent electromagnetic force on the volume induced by the electric and magnetic fields;
an electronic circuit to perform time-gating of an output of the acoustic transducer in temporal correlation with time-dependent adjustments to the magnetic field intensity maximum produced by the adjustable magnetic field generator; and
an electronic controller to control operation of the adjustable magnetic field generator, the electric current source, and the time-gating electronic circuit.

2-13. (canceled)

14. The system of claim 1, wherein the volume comprises biological tissue at least partially encased in bone material.

15-17. (canceled)

18. The system of claim 1, wherein the volume is at least partially encased in a metal-rich boundary.

19-21. (canceled)

22. The system of claim 1, wherein the at least one of the plurality of cross-sections is defined as coplanar relative to the adjustable magnetic field generator.

23. The system of claim 1, wherein the electronic controller synchronizes operation of the adjustable magnetic field generator, the electric current source, and the time-gating electronic circuit.

24-27. (canceled)

28. The system of claim 1, wherein the adjustable magnetic field generator produces a quasistatic magnetic field, and
wherein the electric current source comprises an alternating electric current source modulated at frequencies greater than 20 kilohertz.

29. The system of claim 28, wherein the alternating electric current source is modulated at frequencies greater than 1 megahertz.

30. The system of claim 28, wherein the alternating electric current source is modulated at a frequency corresponding to a target frequency at which to receive acoustic signals via the acoustic transducer.

31. The system of claim 28, wherein the alternating electric current source is modulated at a frequency at least ten times greater than the quasistatic magnetic field.

32-36. (canceled)

37. The system of claim 28, wherein the adjustable magnetic field generator comprises an adjustable array of static magnetic field sources.

38. The system of claim 37, wherein the adjustable array of static magnetic field sources is an array of electromechanically-actuated static magnetic field sources.

39. (canceled)

40. The system of claim 38, wherein the electromechanically-actuated static magnetic field sources are physically rotated during electromechanical actuation to achieve magnetic field amplitude, direction, or polarity modulation.

41-44. (canceled)

45. The system of claim 1, wherein the adjustable magnetic field generator produces alternating magnetic field at a frequency greater than 20 kilohertz.

46. The system of claim 45, wherein the electric current source is a DC current source to generate a DC electric field within the volume.

47. The system of claim 45, wherein the alternating magnetic field is at a frequency greater than 1 megahertz.

48. The system of claim 45, wherein the electric current source is an alternating current source to generate an alternating electric field within the volume.

49. The method of claim 48, wherein the AC electric current source is modulated at a frequency at least two times greater than that of the alternating magnetic field.

50. The system of claim 45, wherein the electric current source is a radio frequency (RF) current source to generate an RF electric field within the volume.

51-55. (canceled)

56. The system of claim 45, wherein the adjustable magnetic field generator comprises a magnetic metamaterial superlens and at least one magnetic field source proximate the superlens.

57-70. (canceled)

71. The system of claim 1, further comprising:
an ultrasonic imaging system to process the acoustic signals received via the acoustic transducer to generate an image of at least a portion of the at least one cross-section.

72. An acoustic-electromagnetic volumetric imaging system, comprising:
a magnetic field source to produce a magnetic field;
a magnetic metamaterial superlens to focus the magnetic field to produce a magnetic field intensity maximum in at least one cross-section of a volume to be imaged;
an electric current source to generate an electric field within the volume;
an acoustic transducer to receive acoustic signals produced within the volume by a time-dependent electromagnetic force on the volume induced by the electric and magnetic fields;
an electronic circuit to perform time-gating of an output of the acoustic transducer in temporal correlation with time-dependent adjustments to the magnetic field intensity maximum; and
an electronic controller to control operation of the electric current source, the time-gating electronic circuit, and at least one of the magnetic field source and the superlens.

73-91. (canceled)

92. The system of claim 72, wherein the at least one of the plurality of cross-sections is defined coplanar relative to the superlens.

93. The system of claim 72, wherein the time-dependent electromagnetic force on the volume comprises a Lorentz force.

94. The system of claim 72, wherein the magnetic field intensity maximum is a hotspot that decays exponentially with respect to distance from the superlens.

95. The system of claim 72, wherein the acoustic transducer comprises an ultrasound microphone.

96. The system of claim 72, wherein the acoustic transducer comprises an array of ultrasound receivers.

97. The system of claim 72, wherein the magnetic field source produces alternating magnetic field modulated at frequencies greater than 20 kilohertz.

98-99. (canceled)

100. The system of claim 72, wherein the electric current source is an alternating current source to generate an alternating electric field within the volume.

101-107. (canceled)

108. The system of claim 72, wherein the magnetic metamaterial superlens comprises magnetic metamaterial with a negative magnetic permeability for at least one polarization of magnetic field.

109. The system of claim 108, wherein the magnetic permeability is negative for all polarizations of magnetic field.

110. The system of claim 109, wherein the magnetic permeability is isotropic and negative for all polarizations of the magnetic field.

111. The system of claim 110, wherein the magnetic permeability is approximately negative one (−1).

112-121. (canceled)

122. A method comprising:
producing, via a magnetic field generator, a magnetic field intensity maximum in at least one cross-section of a volume to be imaged;
generating, via an electric current source, an electric field within the volume;
receiving, via an acoustic transducer, acoustic signals produced within the volume by time-dependent electromagnetic force on the volume induced by the electric and magnetic fields;
time-gating, via an electronic circuit, an output of the acoustic transducer in temporal correlation with time-dependent adjustments to the magnetic field intensity maximum; and
controlling, via an electronic controller, the magnetic field generator, the electric current source, and the electronic circuit.

123-141. (canceled)

142. The method of claim 122, wherein the at least one of the plurality of cross-sections is defined coplanar relative to the magnetic field generator.

143. (canceled)

144. The method of claim 122, wherein the time-dependent electromagnetic force on the volume comprises a Lorentz force.

145-149. (canceled)

150. The method of claim 122, wherein producing the magnetic field intensity maximum comprises producing a quasistatic magnetic field, and
wherein generating the electric field within the volume comprises generating an electric field via an alternating electric current modulated at frequencies greater than 20 kilohertz.

151-158. (canceled)

159. The method of claim 150, wherein the magnetic field generator comprises an adjustable array of static magnetic field sources.

160-166. (canceled)

167. The method of claim 122, wherein producing the magnetic field intensity maximum comprises producing an alternating magnetic field alternating at a frequency greater than 20 kilohertz.

168-169. (canceled)

170. The method of claim 167, wherein generating the electric field comprises generating an AC electric field within the volume via an AC electric current source.

171. (canceled)

172. The method of claim 170, wherein the AC electric current source is a radio frequency (RF) current source.

173-177. (canceled)

178. The method of claim 167, wherein the magnetic field generator comprises a magnetic metamaterial superlens and at least one magnetic field source proximate the superlens.

179-184. (canceled)

185. The method of claim 178, wherein the at least one magnetic field source proximate the superlens comprises a single magnetic field source.

186-192. (canceled)

193. The method of claim 178, wherein magnetic metamaterial of the magnetic metamaterial superlens is a tunable metamaterial.

194-197. (canceled)
